(19) United States
(12) Patent Application Publication
(10) Pub. No.: US 2016/0206081 A1
(43) Pub. Date: Jul. 21, 2016

(54) Tissue Attachment Device And Method

(71) Applicant: Zift Medical, LLC, Excelsior, MN (US)

(72) Inventors: Robert S. Schwartz, Inver Grove Heights, MN (US); Stanton J. Rowe, Newport Coast, CA (US); Robert A. Van Tassel, Excelsior, MN (US); Ralph Schneider, Trabuco Canyon, CA (US); Ming Wu, Tustin, CA (US); David John Blaeser, Brooklyn Park, MN (US); Philip Jon Haarstad, Chanhassen, MN (US); Eric J. Simso, Minnetrista, MN (US); Douglas Jay Krone, Rogers, MN (US); Brian Zelickson, Minneapolis, MN (US); Robert A. Ganz, Minnetonka, MN (US)

(73) Assignee: ZIFT MEDICAL, LLC, Excelsior, MN (US)

(21) Appl. No.: 14/941,429

(22) Filed: Nov. 13, 2015

Related U.S. Application Data
(62) Division of application No. 13/961,785, filed on Aug. 7, 2013.
(60) Provisional application No. 61/680,663, filed on Aug. 7, 2012.

(51) Int. Cl. A45D 44/22; A61B 17/068; A61B 17/064

(52) U.S. Cl. CPC A45D 44/22; A61B 17/064; A61B 17/068; A61B 2017/00004

(57) ABSTRACT

A method and apparatus for attaching tissue to bone in a shifted position without requiring surgical detachment of muscle or connective tissue joining the tissue layer to the bone layer. The skin layer is gently pulled in a non-surgical manner and a fastener of the invention is driven through the skin layer into the bone layer to effect a “skin tightening” procedure.

### RELATED APPLICATIONS

This application is a divisional of and claims priority to U.S. patent application Ser. No. 13/961,785 filed Aug. 7, 2013 entitled Tissue Attachment Device And Method, which claims priority to U.S. Provisional Application Ser. No. 61/680,663 filed Aug. 7, 2012 entitled Tissue Attachment Device And Method, both of which are hereby incorporated herein by reference in their entireties.

### FIELD OF THE INVENTION

This invention generally relates to a method and apparatus for tissue attachment, with one or more tissue layers being attached to bone or other tissue layers. There are multiple applications, including but not limited to dermatology/plastic surgery among others. The application specifically described in this disclosure is to move the skin and surrounding tissues, and fashion of the face or any other body part in relationship to the bone or cartilage structures below, thus effecting a “skin tightening” procedure.

### BACKGROUND OF THE INVENTION

The current standard facelift procedure (technically known as a rhytidectomy) involves the surgical removal of excess skin and tissue from the face and the redraping of the remaining skin on the face and neck. The procedure involves making an incision in front of the ear, which extends up into the hairline and wraps around the bottom of the ear and behind it, ending in the hairline on the back of the neck. After the incision is made, the skin is separated from the tissue underneath, the underlying tissue is sometimes tightened, and the skin is redraped, with the excess skin being removed.

The procedure is expensive, involves a long and painful recovery period, and is accompanied by complications such as infection, bleeding, and those which can accompany anesthesia. In addition, the visual outcome of the procedure can often look unnatural because large areas of tissue must be moved at once.

Efforts have been directed lately toward various techniques that involve fixation of skin through the use of resorbable tacks to effect facial rejuvenation. However, like a traditional facelift, the procedure still involves the use of incisions behind the hairline, requiring a substantial recovery period, and the procedure is only used to correct aging of the top third of the face. There is thus a need in dermatology and plastic surgery for a less invasive, less expensive method or reducing the appearance of wrinkles and excess skin on the face and other areas of the body.

### SUMMARY OF THE INVENTION

Several embodiments are shown and described herein directed to percutaneously lifting, translating, and ‘tightening’ the skin, effecting a reduction in the appearance of wrinkles and excess skin on the face and other areas of the body. The method of the present invention enables a minimally invasive procedure for correcting aging of the face through “skin tightening.” The methods and devices are generally directed to accomplishing the steps of moving the soft tissue layer to a new position in relation to the bone or cartilage below; having a device which is either pushed or ballistically and dynamically driven into the deep layer through a superficial soft tissue layer, thereby holding the soft tissue layer in the new position in relation to the bone or cartilage through the use of anchors.

For example, one embodiment of the method of the invention involves using a delivery device that causes anchors to be impelled at high velocities into the deep layer such as bone whereby they are stopped after a certain distance by the hard layer and become firmly lodged in that layer. The delivery device may be a handheld resembling a pen that has energy capability to accelerate the anchor into the deeper tissues.

In another embodiment, the delivery device comprises an adhesive strip with anchors already lodged in the strip and spaced apart from one another. A practitioner may measure the length of adhesive strip needed, apply it directly to desired area of patient's skin, and accelerate the anchors into the tissue individually.

One aspect of the invention provides a method for relocating a skin layer relative to an adjacent bone layer comprising shifting a skin layer relative to an adjacent bone layer from an original position to a shifted position without cutting muscle or connective tissue associated with the skin layer; holding the skin layer in the shifted position while inserting a fastener through the skin layer into the bone layer to prevent the skin layer from reassuming the original position. One or more additional fasteners may be subsequently inserted through the skin layer into the bone layer.

One aspect of the invention includes inserting a fastener through the skin layer into the bone layer comprises inserting a fastener through the skin layer into the bone layer such that an outer-most feature of the fastener is located just below an outer surface of the skin layer.

Another aspect of the invention involves inserting a fastener through the skin layer comprises ballistically inserting a fastener through the skin layer. This may be accomplished by driving the fastener into the bone layer using a spring-loaded driving mechanism. Alternatively, driving the fastener into the bone layer may be done by using a compressed-gas driving mechanism.

One or more embodiments may include a fastener with an open hollow tip. The fastener may also have an anchoring feature proximate a distal end thereof for preventing the fastener from becoming dislodged from the bone layer.

The invention also provides a fastener for use in anchoring a skin layer in a shifted position to a bone layer thereby overcoming lateral and axial forces imparted by the skin layer on the fastener comprising at least one shaft having a distal end and a proximal end, the at least one shaft including: an anchoring feature proximate the distal end; and, a tissue-holding feature proximate the proximal end.

In one aspect, the fastener comprises a plurality of shafts and the tissue holding feature comprises at least one bridge connecting one of the plurality of shafts to another one of the plurality of shafts.

In another aspect, the aforementioned at least one shaft comprises a hollow portion that is open at the distal end.

In another aspect, the anchoring feature comprises at least one barb.

In another aspect, the fastener comprises a bioabsorbable material.

In another aspect, the tissue holding feature comprises at least one protrusion that extends radially from a longitudinal axis of the shaft when the fastener is driven into the bone layer.

In another aspect the fastener includes a stop that limits a depth that the fastener can be driven into the bone layer.

The shaft of one or more of the fasteners of the invention may have a shaft that comprises a hollow portion that is open at the distal end and the stop comprises a closed proximal end of the hollow portion.

The invention also includes device for use in anchoring a skin layer in a shifted position to a bone layer thereby overcoming lateral and axial forces imparted by the skin layer on the fastener comprising at least one shaft having a distal end and a proximal end comprising: a delivery mechanism; and, at least one fastener; the delivery mechanism including: a barrel; a driving mechanism that drives the at least one mechanism out of a distal end of the barrel; the at least one fastener including at least one shaft having: an anchoring feature proximate the distal end; and, a tissue holding feature proximate the proximal end.

The delivery mechanism may further include a cartridge containing a plurality of the fasteners.

The driving mechanism may comprise a spring; a piston driven by the spring and having a distal end that transfers an axial force released by the spring onto a proximal end of the fastener; a triggering mechanism for releasing energy stored in the spring.

The driving mechanism may also comprise: a capsule containing pressurized gas; a piston driven by the pressurized gas and having a distal end that transfers an axial force onto a proximal end of the fastener; and a triggering mechanism for releasing the axial force stored in the spring.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIGS. 1a-c show the manner in which tissue is relocated using the present invention;

FIG. 2 is a cutaway view of an fastener of the present invention embedded into tissue;

FIGS. 3a-b show a patients face before and after wrinkles have been removed using the present invention;

FIG. 4 is an elevation of an embodiment of an fastener of the present invention;

FIG. 5 is an elevation of an embodiment of an fastener of the present invention;

FIG. 6 is a perspective view of an embodiment of an fastener of the present invention;

FIG. 7 is an elevation of an embodiment of an fastener of the present invention;

FIGS. 8a-b are elevations of an embodiment of an fastener of the present invention;

FIG. 9 shows an embodiment an fastener of the present invention being driven into bone;

FIG. 10 is a perspective view of an embodiment of a fastener of the present invention.

FIG. 11a is a perspective view of an embodiment of a fastener of the present invention.

FIG. 11b is a transparent perspective view of the embodiment of a fastener of the present invention shown in FIG. 11a.

FIG. 12 is a perspective view of an embodiment of a fastener of the present invention.

FIG. 13 is a perspective view of an embodiment of a fastener of the present invention.

FIG. 14 is a perspective view of an embodiment of a fastener of the present invention.

FIG. 15 is a perspective view of an embodiment of a fastener of the present invention.

FIG. 16 is a perspective view of an embodiment of a fastener of the present invention.

FIG. 17 is a perspective view of an embodiment of a fastener of the present invention.

FIG. 18 is a perspective view of an embodiment of a fastener of the present invention.

FIG. 19 is a perspective view of an embodiment of a fastener of the present invention.

FIG. 20 is a perspective view of the embodiment of a fastener of FIG. 19 loaded into a distal end of a delivery device.

FIG. 21 is a perspective view of an embodiment of a delivery device of the present invention;

FIG. 22 is a perspective view of an embodiment of a delivery device of the present invention;

FIG. 23 is a perspective view of an embodiment of a delivery device of the present invention; and

FIG. 24 is a perspective view of an embodiment of a delivery device of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

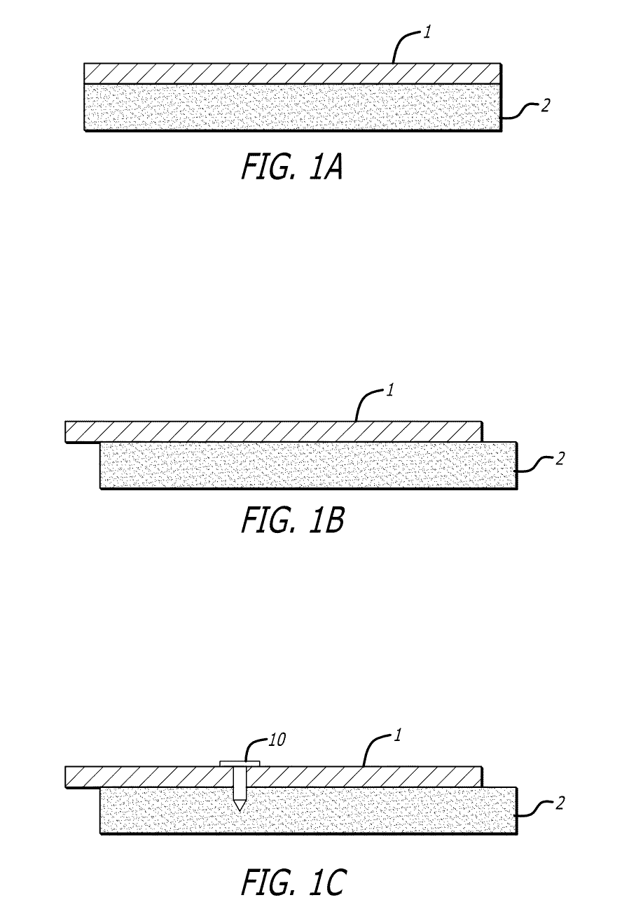

Referring now to the figures, FIG. 1 illustrates the general method of the present invention. As shown in FIG. 1a, the method begins with a selection of a skin layer 1 to be relocated relative to a bone or cartilage layer 2. In FIG. 1b, the skin layer 1 has been shifted relative to the bone layer 2. No connective tissue between the skin layer 1 and the bone layer 2 has been severed or damaged. The tissue 1 has simply been relocated using gentle pressure, such as by a finger. Most procedures will attain desirable results with only a small shifting, akin to taking one's finger and moving one's forehead skin around gently. It is noted that the discontinuity in the edges of the tissue sample shown in FIG. 1b are simply provided for illustrative purposes to show that the skin layer 1 has been shifted relative to the bone layer 2 and is not to be interpreted as a severing or slicing of the selected layer 1. In actuality, the continuous skin layer 1 stretches, and thus releasing the skin layer 1 would result in its return to the original position shown in FIG. 1a.

In order to prevent the return of the skin layer 1 to its original position relative to the bone or cartilage layer 2 of FIG. 1a, a fastener 10 of the present invention is used to affix the skin layer 1 to the bone or cartilage layer 2 at its new position. This step is shown in FIG. 1c. The fastener 10 is shown as a simply tack-like device in FIG. 1c. Various embodiments of fastener 10 will be shown and described below.

The method described above and shown in FIG. 1 is a simple illustration of the basic concepts of the present invention. It is envisioned that the steps of shifting the skin and anchoring it a new position will be repeated many times until a desired result is achieved. It is also likely that a single shift will be followed by multiple fasteners 10 be applied to secure a given shift of the skin layer 1. Subsequent shifts will then likely be required in order to achieve a desired, natural-looking result.

In one embodiment of the method of the present invention, extremely small, short and shallow incisions may be made at the site where the fastener 10 is being driven such that the head or top of the fastener rests just below the surface of the skin such that the fastener remains hidden. These incisions are so small that often no bleeding occurs. Positive results have been attained with incisions that are no longer than 0.05 inches.

In order to accommodate the insertion of several fasteners 10, various delivery mechanisms are described below, some of which allow the delivery of rapidly successive fasteners, in the spirit of tools found in carpentry, such as nail guns, staple guns, and the like. Other delivery mechanism described below allow for the delivery of several fasteners simultaneously.

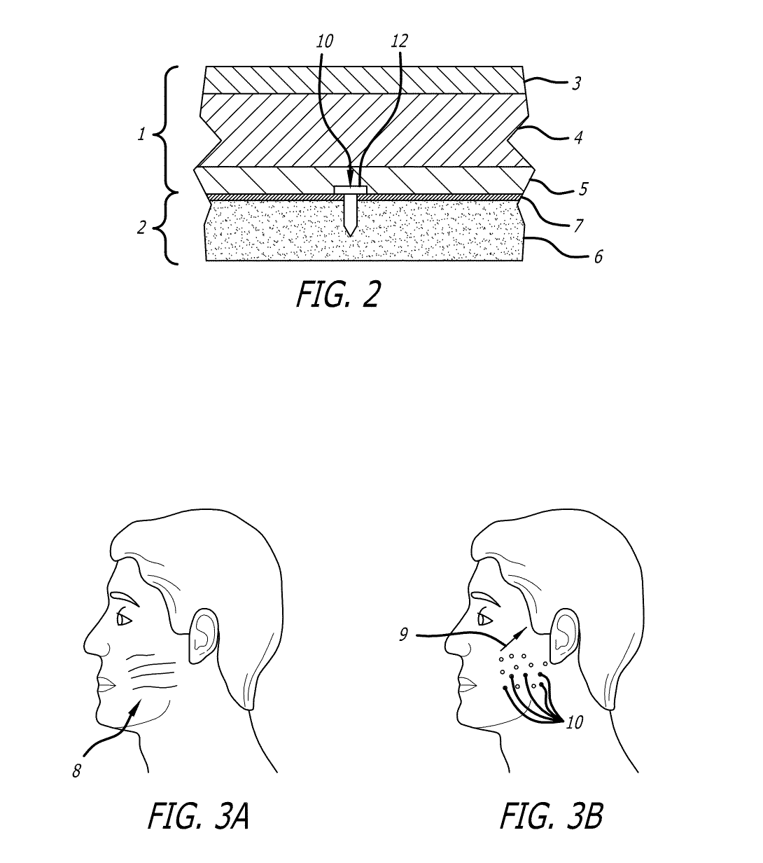

FIG. 2 provides a more detailed view of the skin layer 1, the bone or cartilage layer 2 and the device 10. The skin layer 1 comprises an epidermis 3, a dermis 4 and a subcutaneous layer 5. Superficial musculoaponeurotic system (SMAS) fascia is a fanlike fascia that envelops the face and provides a suspensory sheet which distributes forces of facial expression. On a cellular level it comprised of collagen fibers, elastic fibers, fat cells and muscle fibers.

The bone layer 2 includes the bone 6 and a periosteum 7. It can be seen in FIG. 2 that the fastener 10 is implanted such that it penetrates the bone 6 and the periosteum 7 and the proximal end 12 of the fastener 10 terminates within the subcutaneous layer 5, or the dermis 4. In this way, the fastener 10 remains invisible once implanted. The SMAS layer is the structure upon all modern, traditional facelifts are based. Traditional facelift addresses the lower face and neck. It involves an incision made in the hairline, starting above the ear, continuing behind the ear, curving around the ear and ending in the hairline behind the ear. The surgeon dissects the skin from the underlying fat and muscle. He will then use sutures to lift and reposition the muscle layer (“SMAS”—superficial musculo-aponeurotic system) toward the ears. This muscle tightening is thought to provide longevity to the surgical result. Next, the excess skin is removed and the incisions are closed.

FIG. 3 shows the general effect of correcting the appearance of wrinkles 8 on the face (FIG. 3a) through the use of multiple anchors 10 to hold the soft tissue in place in a new position after the face has been shifted in the direction of arrow 9 (FIG. 3b).

Fasteners

Turning now to the fasteners 10 of the invention, the fasteners 10 may take one of many possible forms. Generally, they may be circular, flat, or any other configuration geometrically that allows them to penetrate the bone or cartilage with a sharpened distal end. The device may be textured on the surface, for example with a micro-texturing that allows cells to more easily attach and anchors the device permanently in the bone or cartilage. The anchors may be metallic or they may be polymeric. They may be a combination of metal and polymer. The polymer may be biostable or bioabsorbable. It may contain drugs for elution. The anchors may be electrically conductive and may permit electrical energy for either energy delivery or energy recording of biologic signals. Examples of embodiments having various characteristics are shown in the Figures and are not meant to be limiting. It is to be understood that any of the characteristics may be incorporated into any of the embodiments of the invention.

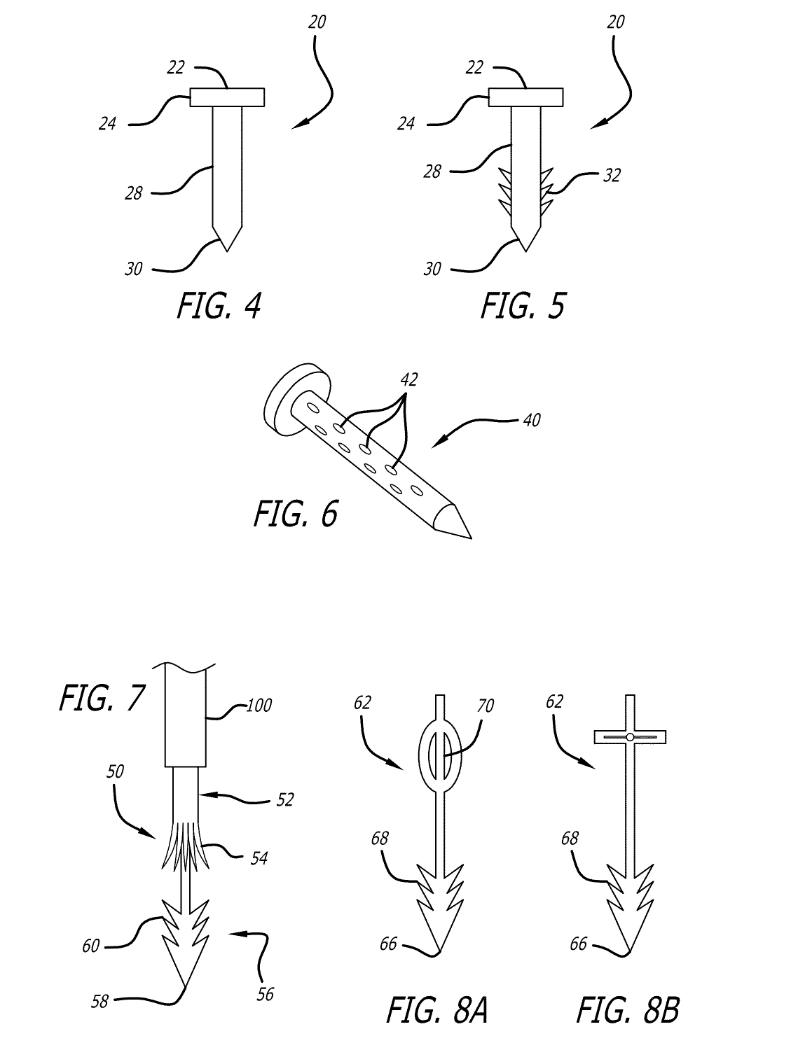

Generally, the fasteners include at least one shaft that includes an anchoring feature proximate the distal end of the shaft and a tissue-holding feature proximate the proximal end of the shaft. A first embodiment 20 of a fastener 10 is shown in FIG. 4. This embodiment 20 includes a head 22 as a tissue-holding feature at a proximal end 24 of the anchor 20. The head 22 may be circular and generally flat for pushing or hammering. A shaft 28 is provided with sufficient length to anchor the fastener 20 deep enough into the bone to resist becoming dislodged, while still terminating such that the head results at a desired depth in the skin layer 1. The distal end of the shaft presents an anchoring feature that includes a sharpened distal tip 30 that allows the device 20 to be driven through the skin layer 1 into the bone layer 2. As shown in FIG. 5, the anchoring feature may also include features 32 that prevent the device 20 from migrating proximally after having been implanted. These features 32 may comprise hooks, barbs, ridges, high-friction surfaces, in-growth inducing coatings, adhesives, and the like.

FIG. 6 shows an embodiment 40 in which the fastener 10 has a hollow shaft that includes apertures 42 that lead to an interior lumen that may contain a drug or agent. The drugs may elute out slowly or rapidly and may be initially contained within the lumen, or may be injected through the lumen after the device is implanted. The drug may have a polymer component to it to program the rate of delivery. The drug may be in the form of nanoparticles that are driven into the bone or leach out into the bone or cartilage at a programmable rate.

FIG. 7 shows an embodiment of a fastener 50 that includes plungers 52 and 54 that engage the tissue and anchor the tissue to the bone or cartilage. In FIG. 7, the device 50 is being deployed from a delivery device 100. The device 50 includes a tissue-holding feature shown as a first plunger 52 having a plurality of fingers 54 that splay outwardly as they exit the delivery device 100 and engage tissue of the skin layer 1. A second plunger 56 having a sharp distal tip 58 and anchoring features 60 extends partly through the first plunger and is designed to penetrate the bone layer 2 and remain anchored therein.

FIGS. 8a and 8b show a fastener embodiment 62 in which the two plungers are connected as one device. In embodiment 62, the device includes a distal end 64 with a sharpened tip 66 and anchoring features 68, similar to those of the embodiment 50 of FIG. 7. The tissue-holding feature at the proximal end of the device 62, however, includes a plurality of slits 70 that, when compressed or foreshortened, flare outwardly to engage tissue, as seen in FIG. 8b.

The slots 70 may be actively flared in a subsequent step, however, it is envisioned that such slots 70 may be advantageously paired with one or more of the ballisitic delivery device embodiments, described below. When the device 10 is driven into the bone layer, there is substantial compression on the device that causes the precut slots 70 to expand radially, markedly increasing the cross-sectional area of a small section of the anchor. When placed appropriately, this expanding tissue-holding concept will serve to grasp the soft tissue ends spreading the stress and strain on the tissue out over a larger area to prevent laceration. It will serve as a larger tissue-holding feature for the soft tissue because its surface area in contact with the soft tissue is substantially increased.

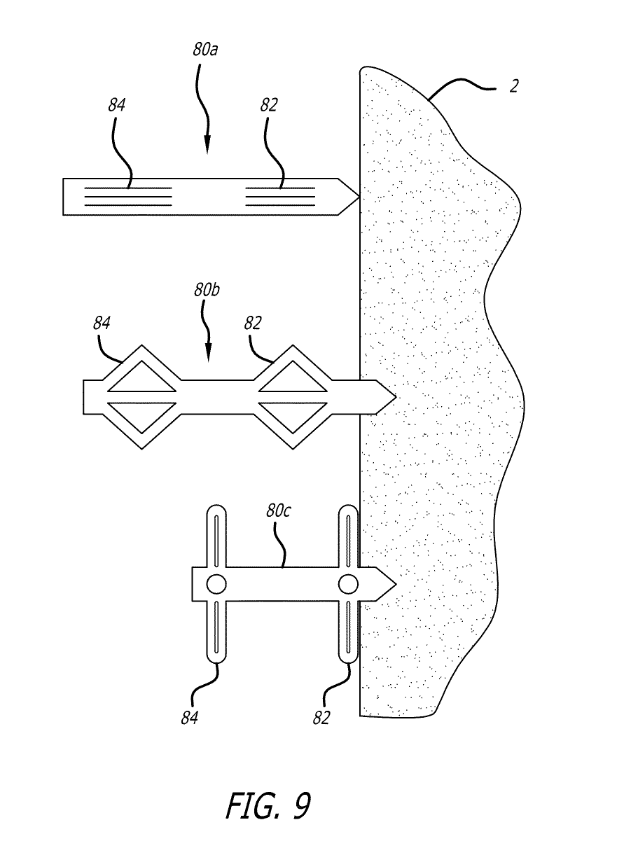

FIG. 9 shows a fastener embodiment 80 incorporating a distal set of slots 82 and a proximal set of slots 84. Three fasteners 80 are shown in various stages of implantation. Device 80a has not yet encountered the bone layer 2 and is therefore not yet subject to compressive force. Device 80b has contacted the bone layer 2 and, due to the compressive force provided by the resistance of the bone layer 2, the slots 82 and 84 begin to bend outwardly. Device 80c is completely driven into the bone layer 2 and both sets of slots 82 and 84 are completely flared. The distal set 82 forms a stop that prevents the device 80 from travelling further into the bone layer 2. The proximal set 84 forms a tissue-holding feature that engages tissue from the tissue layer 1, thereby preventing the tissue layer 1 from sliding over the device 80 and adjusting itself to a previous position. If it is desired to further increase the tissue holding power of the proximal slots 84, additional features such as barbs or hooks could be incorporated into the device 80. Advantageously, this configuration allows a small diameter device 80 to be inserted through the skin, while expansion does not occur until the device reaches the dermal or subcutaneous layer, thereby maximizing holding power while minimizing trauma to the visible entry point, thus promoting quick healing.

The resulting arms formed by the slots may be retractable in the event that the operator is unhappy or unsatisfied with placement, and re-deployed for better configurations and results. Additionally, the above describes only two locations on the anchor where the “stop” components are placed, but multiple other locations may be made as well to further anchor in the soft tissue.

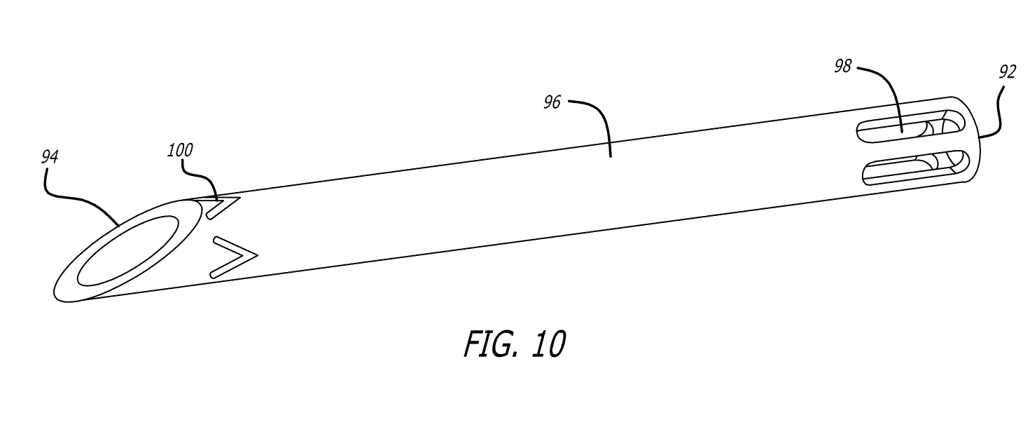

FIG. 10 shows another fastener embodiment 90. Fastener 90 includes a proximal end 92, a distal end 94 and a shaft 96. The proximal end 92 includes a plurality of slots 98, similar to the slots 84 of embodiment 80, described above, in both form and function. Slots 98 allow the material between the slots to flare outwardly to form a tissue-holding feature. The distal end 94 is sharpened at an angle to allow the fastener to pierce bone tissue. An anchoring feature 100 includes a plurality of angled cut-outs that flare outwardly when driven into the bone for grabbing the bone layer. The cut-outs may flare outwardly due to the force of being driven into the bone or they may be formed of a memory metal or they may be manufactured with outward flares. The embodiment 96 is also shown has having a hollow shaft 96 with an open distal end. It has been found that a hollow shaft with an open distal end results in a fastener that is easier to drive into the bone, as less bone material needs to be displaced.

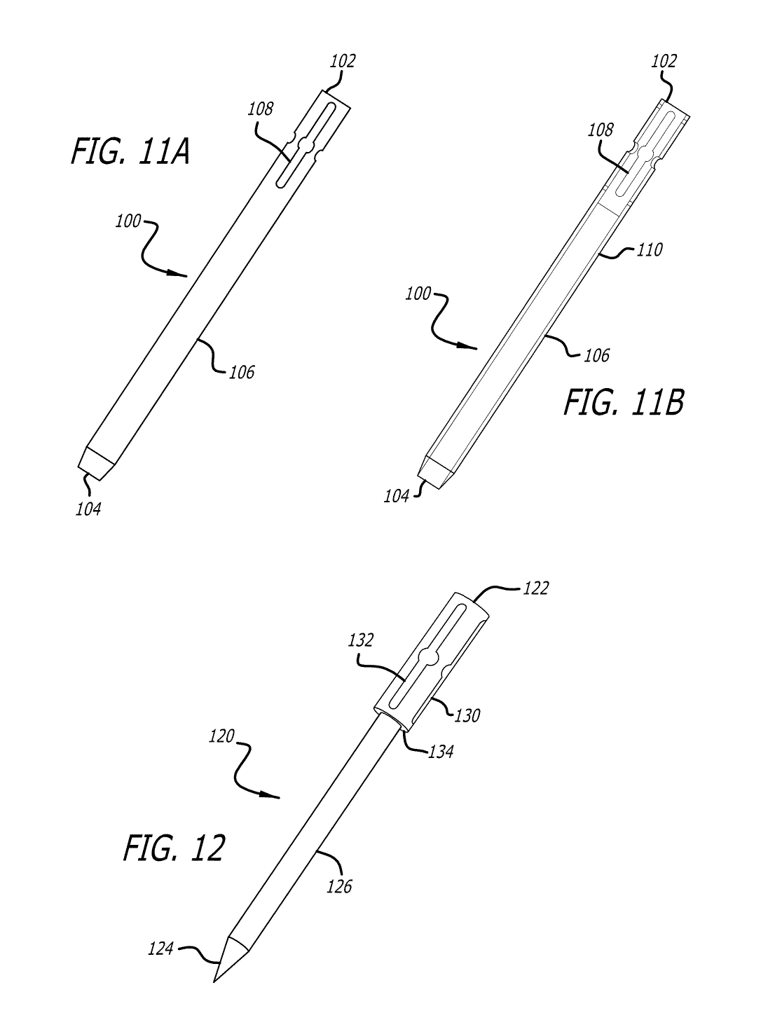

FIGS. 11a and 11b show a fastener embodiment 100 that includes a proximal end 102, a distal end 104 and a shaft 106. The proximal end 102 includes a plurality of slots 108, similar to the slots 84 of embodiment 80, described above, in both form and function. Slots 108 allow the material between the slots to flare outwardly to form a tissue-holding feature. Like fastener 90, the shaft 106 is also hollow. As best seen in FIG. 11b, which is depicted as translucent to show the interior features of the device 100, the interior lumen of the shaft 106 terminates proximally at a stop 110. Stop 110 limits the depth that the fastener 100 can be driven into the bone layer. The stop 110 also prevents inward deflection of the tissue-holding feature formed by the slots 108.

The distal tip 104 of fastener 100 is also sharpened. However, rather than being sharpened at an angle like fastener 90, the distal tip 104 of fastener 100 is sharpened circumferentially to form a beveled edge. It has been found that a tip sharpened this way penetrates bone more easily, possibly due to the cancelation of all side forces resulting from the angled surfaces. Additionally, the angled tip of fastener 90 has a rapidly-increasing surface area as it is being driven into bone. The beveled tip, on the other hand, as a relatively constant surface area contacting the bone as it is being driven. Experimentation has shown that holding all other variables constant, driving 5 mm long fasteners with angled tips into a bone layer with a constant driving force resulted in a driving depth of 2-3 mm. When driving 5 mm fasteners with beveled tips using the same driving force, the driving depth was approximately 4 mm.

It must be emphasized that any of the features described herein with regard to one embodiment may be combined with any of the features of the other embodiments.

FIG. 12 shows yet another fastener embodiment 120. Fastener 120 has a shaft 126 with a proximal end 122, and a distal end 124. The shaft 124 of fastener 120 is shown, by way of example, as being solid and sharpened to a point. The proximal end 122 of the fastener 120 has a tissue-holding feature 130 with a plurality of slots 132 that function in a similar fashion to the slots described above in association with the various other embodiments. The tissue-holding feature 130, however, is shown as having an increased radius in relation to the shaft 126. This is because the shaft 126 is solid, so forming slots in a solid shaft would not result in flaring when the shaft is impacted. Additionally, a distal edge 134 of the tissue-holding feature 130 forms a stop that limits the depth the fastener 120 can be driven into a bone layer. The distal edge 132 also causes outward deflection of the tissue-holding feature formed by the slots 134.

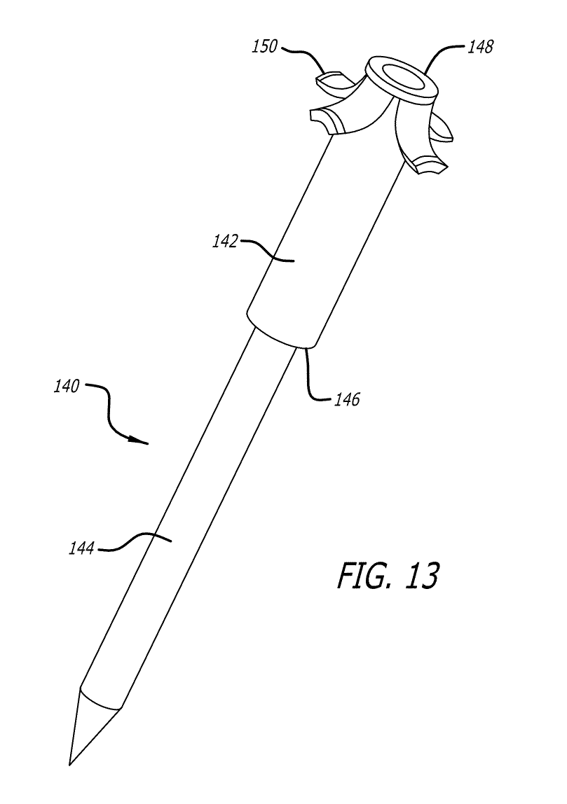

FIGS. 13-9 show various fastener embodiments utilizing different tissue-holding features. For example, FIG. 13 shows a fastener embodiment 140 with a sliding collar 142 that slides over the shaft 144 of the fastener 140. The collar 142 has a distal edge 146 that acts as a stop to limit the depth that the fastener 140 can be driven into a bone layer. The edge 146 also causes the collar 142 to slide proximally over the shaft 144 when the edge 146 contacts the bone layer. Sliding proximally over the shaft 144 causes a proximal edge 148 of the collar to impact and deform a plurality of wings 150. The wings 150 are deformed to splay outwardly, thereby creating a tissue-holding feature.

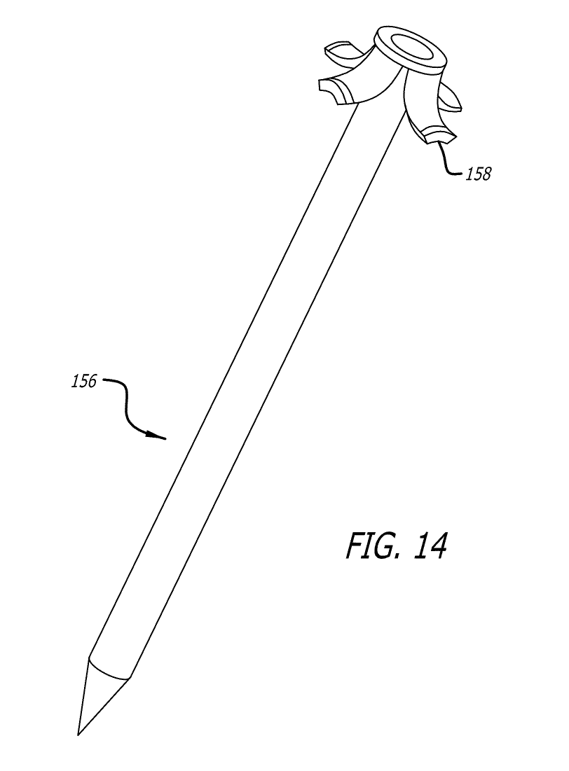

FIG. 14 shows a fastener embodiment 156 having a similar tissue-holding feature including a plurality of outwardly-splayed wings 158. These wings 158, however, are formed of a memory metal such that they splay without requiring impact forces.

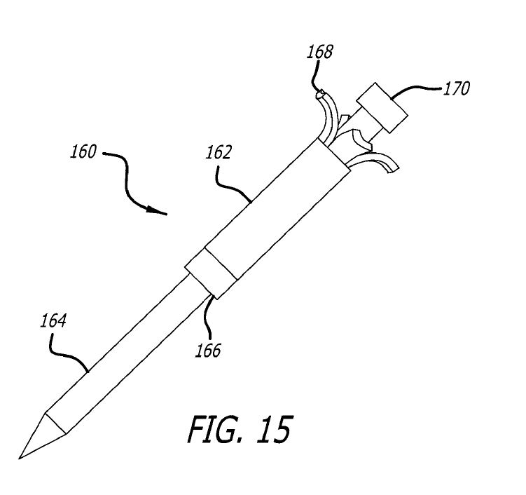

FIG. 15 shows a fastener embodiment 160 with a sliding collar 162 that slides over a shaft 164 of the fastener 160. The embodiment 160 also includes a fixed collar 166 that has a distal edge that acts as a stop to limit the depth that the fastener 160 can be driven into a bone layer. The sliding collar 162 has a plurality of wings 168 at its proximal end. The shaft 164 has a proximal fixed collar 90 at its proximal end, which keeps the sliding collar 162 from sliding too far proximally. The wings 168 splay are made from nitinol or a similar memory metal and splay outwardly when delivered to form a tissue-holding feature.

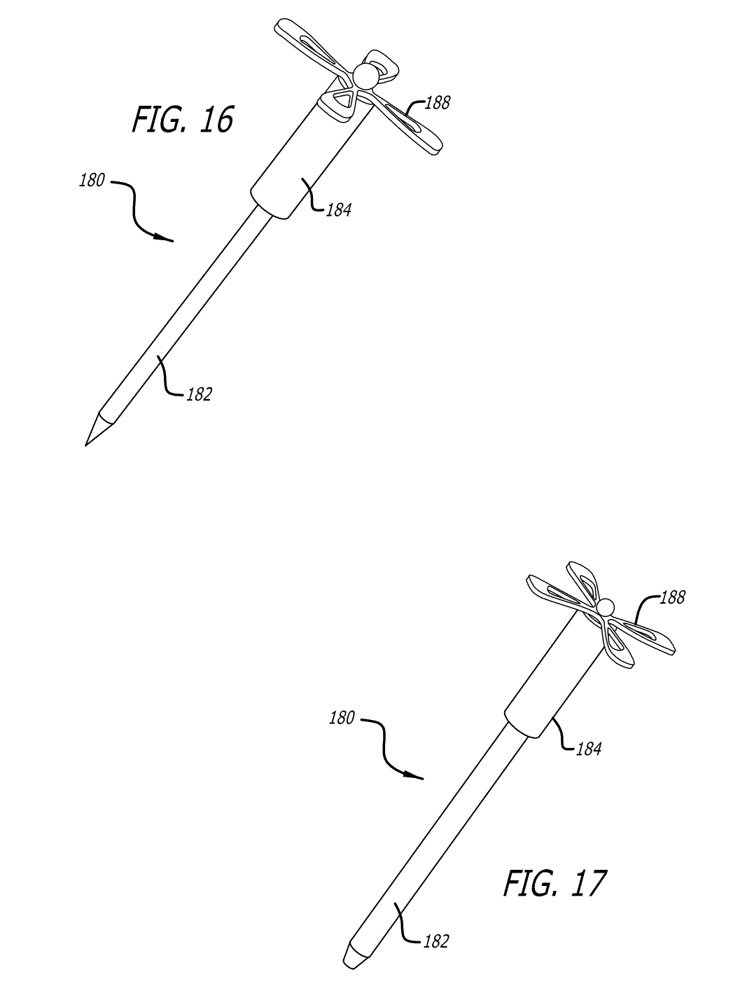

FIG. 16 shows a fastener embodiment 180 with a shaft 182 and a collar 184 having a distal edge 186 that acts as a stop. The fastener 180 includes as a tissue-holding feature a plurality of petals 188 at its proximal end. The petals 188 are flat and radiate outwardly, perpendicular to a longitudinal axis of the shaft 182. The petals 188 may be formed to splay outwardly by way of the collar 184, as described above, or the petals 188 may be formed of memory material. Additionally, the petals 188 may be stored in a delivery device in a proximally or distally directed fashion.

FIG. 17 demonstrates that the shaft 182 of the fastener 180 may be hollow and that each of the embodiments shown has features that may be combined with features shown in other embodiments.

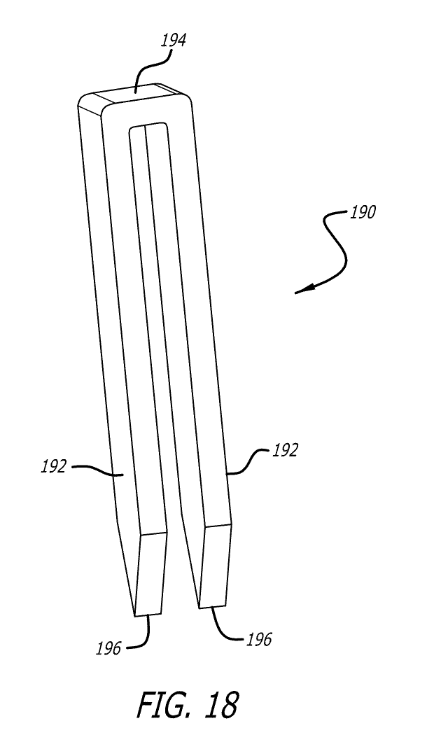

FIG. 18 shows a fastener embodiment 190 that includes two shafts 192 connected at their proximal ends with a bridge 194, thus forming a staple. The bridge 194 serves as a tissue-holding feature. Each of the shafts 192 includes a pointed distal tip 196 that allows the fastener 190 to be driven into bone. The shafts 192 serve as anchoring features.

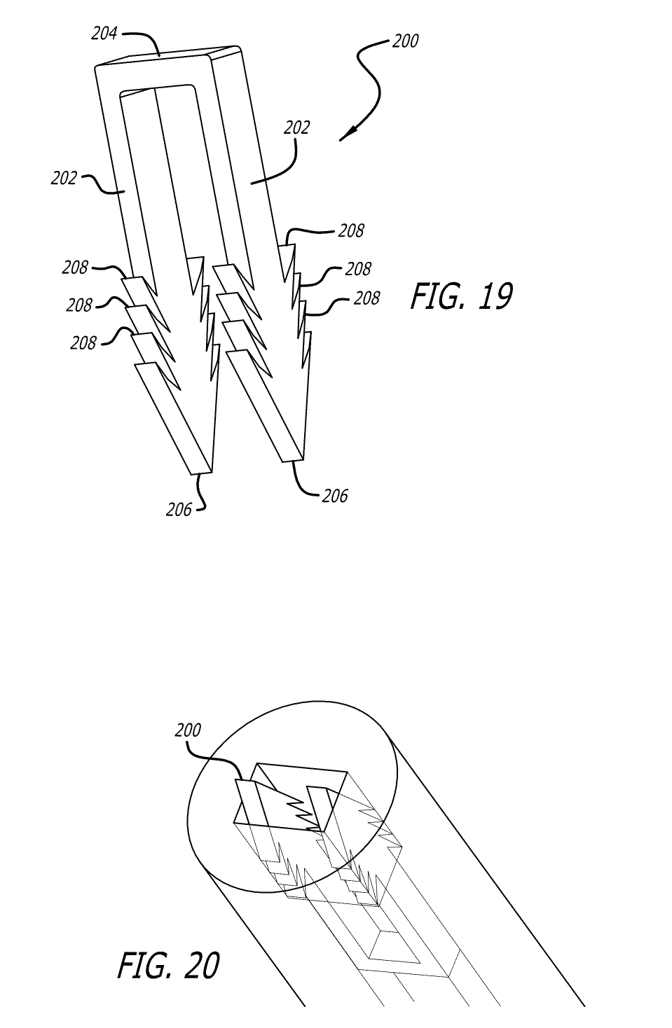

FIG. 19 shows a fastener embodiment 200 that includes two shafts 202 connected at their proximal ends with a bridge 204, thus forming a staple. Like that of FIG. 18, the bridge 204 serves as a tissue-holding feature. Each of the shafts 202 includes a pointed distal tip 206 that allows the fastener 200 to be driven into bone. The shafts 202 also include anchoring features in the form of barbs 208. FIG. 20 shows the fastener 200 loaded into the distal end of a delivery device. The various embodiments of delivery devices are discussed in more detail below.

Delivery Devices

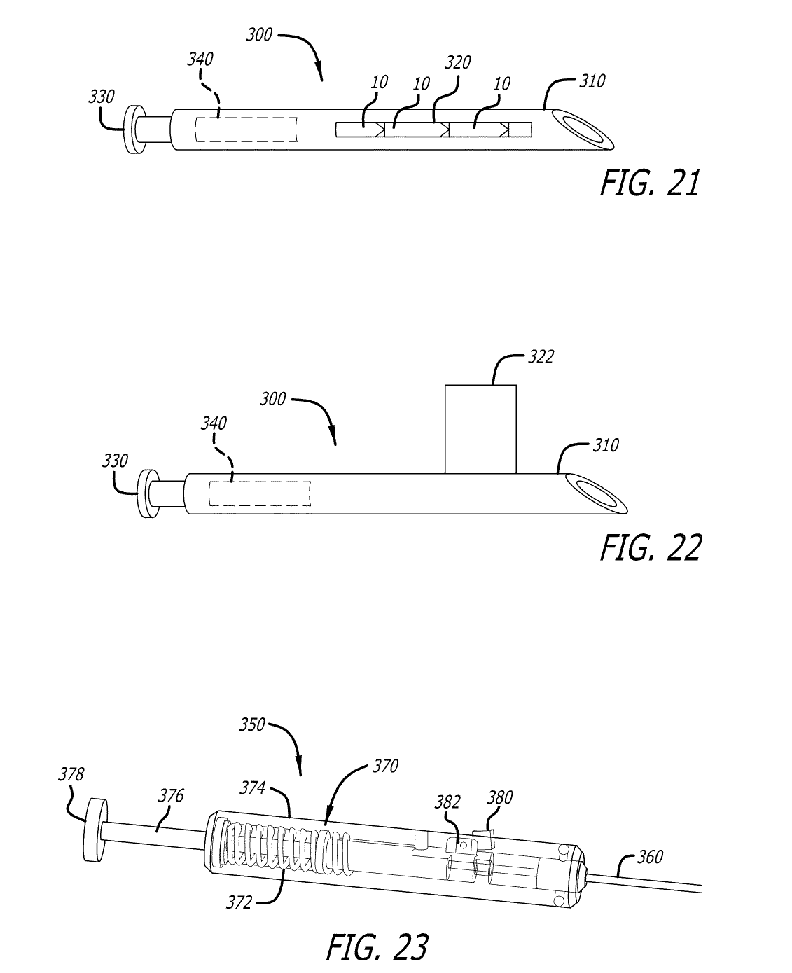

FIGS. 21 and 22 generally show the basic components of a delivery device 300 of the invention. The delivery device includes a hollow needle or barrel 310, a magazine 320 containing one or more anchors 10, a triggering mechanism 330, and a driving mechanism 340.

The barrel 310 may have a sharp tip for making a small incision in the outermost surface of the skin in order to countersink the fastener 10. The barrel also has an interior lumen sized to carry a fastener 10. The magazine 320 may comprise the interior lumen, or may be in the form of a magazine cartridge 322, attachable to the device 300.

The driving mechanism 340 may take on various forms but all are designed to impart an axial force onto the fastener 10. Driving forces that may be useful include pneumatic, spring, electromagnetic, compressed gas such as carbon dioxide, acoustic, ultrasonic, hydraulic impulsion, and the like.

The trigger mechanism 330 is a mechanism used to release or activate the driving force. The release mechanism may be a push button, such as that shown in FIGS. 21 and 22, or may be a finger trigger, foot pedal, bulb, etc.

A more detailed delivery device embodiment 350 is shown in FIG. 23. The delivery device 350 includes a barrel 360, a driving mechanism 370, and a trigger mechanism 380. The driving mechanism 370 includes a spring 372 connected at a distal end to a body 374 and at a proximal end to a piston 376. The trigger mechanism 380 is located on the side of the body and includes a catch 382 that pivots into an interference position with a portion of the piston 376.

In operation, the operator loads a fastener into a lumen of the barrel 360. Alternatively, the fastener is pre-loaded or a magazine is provided with a plurality of fasteners loaded therein. The operator then pulls the proximal end 378 of the piston 376 in a proximal direction. This stretches or compresses the spring 372, depending on how the spring is loaded into the device, providing potential energy for driving the piston 376. The piston 376 is pulled until the catch 382 of the trigger mechanism 380 pivots into an interference position with the distal end of the piston. The delivery device 350 is now loaded and cocked. The operator now gently pulls the skin of a patient or procedure recipient into a desired position, thus shifting the skin layer relative to the bone layer, and holds the skin in the shifted position with his or her finger. The barrel 360 is then pointed at a target site, possibly breaking the skin slightly, and the trigger mechanism 380 is depressed, which pivots the catch 382 out of the interference position, releasing the piston 376. The spring 372 releases its stored energy, and drives the piston axially into a proximal end of a fastener 10, driving the fastener 10 into a bone layer.

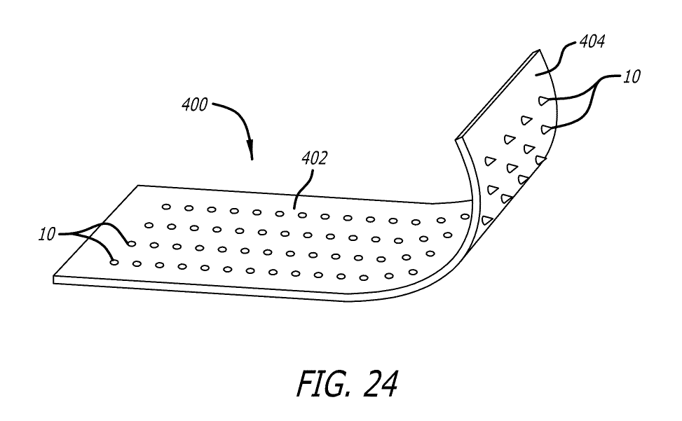

Another embodiment 400 of a delivery device is shown in FIG. 24. Delivery device 400 includes a tape-like substrate having a first side 402 and a second side 404. A plurality of anchors 10 are manufactured into the substrate such that the distal ends of the anchors 10 extend through the second side 404. The second side may include a mild adhesive.

Using the delivery device 400 involves cutting a desired size and shape of the substrate appropriate for the placement of the anchors 10. The targeted skin is appropriately numbed and the substrate 400 is placed on the skin. The adhesive ensures that the substrate will not become accidentally relocated prior to the implantation of the anchors 10. The skin is then shifted to a desired new location and a rubber mallet, or the like, is used to gently tap the anchors 10 into place. Because the anchors 10 are very small, each blow of the mallet contacts several, if not all of the anchors 10, thus requiring few blows. After the anchors 10 are embedded, the substrate 400 is simply peeled away from the patient, leaving the anchors 10 in place.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A fastener for use in anchoring a skin layer in a shifted position to a bone layer thereby overcoming lateral and axial forces imparted by said skin layer on said fastener comprising at least one shaft having a distal end and a proximal end, said at least one shaft including:
an anchoring feature proximate said distal end; and,
a tissue-holding feature proximate said proximal end.

2. The fastener of claim 1 wherein said at least one shaft comprises a plurality of shafts and said tissue holding feature comprises at least one bridge connecting one of said plurality of shafts to another one of said plurality of shafts.

3. The fastener of claim 1 wherein said at least one shaft comprises an hollow portion that is open at said distal end.

4. The fastener of claim 1 wherein said anchoring feature comprises at least one barb.

5. The fastener of claim 1 wherein said fastener comprises a bioabsorbable material.

6. The fastener of claim 1 wherein said tissue holding feature comprises at least one protrusion that extends radially from a longitudinal axis of said shaft when said fastener is driven into said bone layer.

7. The fastener of claim 1 further comprising a stop that limits a depth that said fastener can be driven into said bone layer.

8. The fastener of claim 7 wherein said shaft comprises a hollow portion that is open at said distal end and said stop comprises a closed proximal end of said hollow portion.

9. A device for use in anchoring a skin layer in a shifted position relative to a bone layer thereby overcoming lateral and axial forces imparted by said skin layer on said fastener comprising at least one shaft having a distal end and a proximal end comprising:
a delivery mechanism; and,
at least one fastener;
said delivery mechanism including:
a barrel;
a driving mechanism that drives said at least one mechanism out of a distal end of said barrel;
said at least one fastener including at least one shaft having:
an anchoring feature proximate said distal end; and,
a tissue holding feature proximate said proximal end.

10. The device of claim 9 wherein said delivery mechanism further includes a cartridge containing a plurality of said fasteners.

11. The device of claim 9 wherein said driving mechanism comprises:
a spring;
a piston driven by said spring and having a distal end that transfers an axial force released by said spring onto a proximal end of said fastener;
a triggering mechanism for releasing energy stored in said spring.

12. The device of claim 9 wherein said driving mechanism comprises:
a capsule containing pressurized gas;
a piston driven by said pressurized gas and having a distal end that transfers an axial force onto a proximal end of said fastener;
a triggering mechanism for releasing said axial force stored in said spring.
